(19) United States
(12) Patent Application Publication
(10) Pub. No.: US 2009/0105451 A1
(43) Pub. Date: Apr. 23, 2009

(54) Use of aqueous wettable hydrophobic chromatographic media for the purification of peptides, and other biomolecules

(76) Inventors: Ashok K. Shukla, Ellicott City, MD (US); Mukta M. Shukla, Ellicott City, MD (US)

Correspondence Address: ASHOK K. SHUKLA, 10316 KINGWAY COURT, ELLICOTT CITY, MD, 21042, US

(21) Appl. No.: 12/288,707

(22) Filed: Oct. 22, 2008

Related U.S. Application Data
(60) Provisional application No. 60/999,725, filed on Oct. 22, 2007.

(51) Int. Cl. C07K 1/18

(52) U.S. Cl. 530/344

(57) ABSTRACT

The use of hydrophobic coated metal oxides particles or film for the purifications of proteins, peptides or other biomolecules. This process eliminates two steps of purification as compared to silica or polymer based particles.

This application (Applicants) claims the benefit of priority date Oct. 22, 2007 of provisional application No. 60/999,725.

### FIELD OF THE INVENTION

In chromatography—a method for separation of biomolecules; the biomolecules are attached to the surface of the chromatographic particles and conditioned with one buffer or solvent and eluted after wash with a different buffer or solvent. Most common chromatographic method is reverse phase (RP) chromatography. In reversed phase, silica is modified with long chain hydrocarbons such as C-18. In reverse phase, the proteins or peptides are bound in aqueous solution to the RP-media and eluted with the organic solvent containing buffer or solution. One drawback of this method is that the RP-media must be wetted with the organic solution and only then it can be used. To eliminate the two steps of wetting with the organic solvent and conditioning with the aqueous solution, we describe here a new method for the purification of the biomolecules by using modified metal oxides.

### BACKGROUND OF THE INVENTION

Definitions; Here we try to explain the definition of the terms used but this does not limit its vast definition.

Particles: can be porous or non-porous, any shape and size.

Metal oxide: Metal oxides of individual metals or mixed metal oxides, which contain more than one metal. Metal oxides may contain other elements or functional groups.

Modification of metal oxides: means that the surface density of the water binding groups on the metal oxide is reduced by some chemical reactions or by physical means (coating, covering, non covalent interaction).

Biomolecules: The molecule of biological source can be further modified or fragmented. This is not limited to protein, peptides, DNA, RNA, lipids, small molecules, such as vitamins, carbohydrates, oligosacchrides, and combination of these molecules.

The most common methods used for the purifications of proteins and peptides are electrophoresis and chromatography. In chromatographic method, the biomolecules are attached to the surface of the chromatographic particle and conditioned with one buffer or solvent and after wash eluted with a different buffer or solvent. Most common chromatographic method is reverse phase (RP) chromatography. In reversed phase, silica is modified with long chain hydrocarbons such as C-18. In reverse phase, the proteins or peptides are bound in aqueous solution to the RP-media and eluted with the organic solvent containing buffer or solution. One drawback of this method is that the RP-media must be wetted with the organic solution and conditioned with the aqueous phase, before the biomolecules are absorbed on the surface of RP-chromatographic media. This step of wetting is necessary to bind the biomolecules. If the wetting step is eliminated, there is almost no binding of the biomolecules with the RP-media, because the hydrophobic surface repels the water molecule and does not allow the interaction with the biomolecules.

To eliminate the two steps of wetting with the organic solvent and conditioning with the aqueous solution, we describe here a new method for the purification of the biomolecules. The porous metal oxides particles such as TiO2 or ZrO2 or any other stable transition metal oxide are used for the chromatographic applications. The hydrophobic modified metal oxides are easily wettable in aqueous solution and bind the proteins or peptides or other molecules directly to the surface of the particles, without the pre treatment of the organic phase conditioning as described above. Therefore, use of these modified metal oxides will be more selective and efficient than the above RP-media method. Once the desired molecule is attached to the modified metal oxides, it can be purified or eluted with a different buffer, in this case the buffer or solution, contains the organic solvent (for example, methanol, ethanol, acetone, acetonitrile or water miscible organic solvents.

This method with the coated metal oxides can also be used on any surface and not only with the chromatographic particles. These surfaces can also be used in a biochip.

The various features of novelty, which characterize this invention, are pointed out with particularity in the claims annexed to and forming a part of this disclosure. For a better understanding of the invention, its advantages and objects, reference is made to the accompanying drawings and descriptive matter in which a preferred embodiment of the invention is illustrated.

### BRIEF DESCRIPTION OF THE DRAWINGS

FIG. 1. is an expanded view of one embodiment of a metal oxide particle coated with non covalently bound polymer.

### DESCRIPTION OF THE PREFERRED EMBODIMENTS

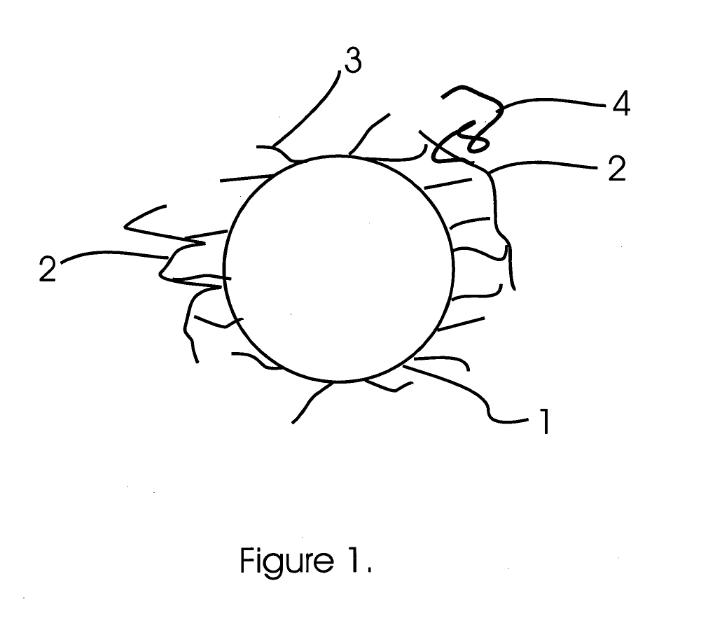

FIG. 1 shows that the metal oxide particle (1) is coated with a polymer (2) and this polymer (2) imparts hydrophobic properties to the particles. This polymer coating density many are random or symmetrical. The water molecules can attach to the (3), which allows a binding of the protein or peptides (4) to the hydrophobic area of the particle or surface. This is a method for the purification and enrichment of biomolecules by using modified metal oxide particles, such particles are hydrophobic and wetable with aqueous solution. The wetable character should be retained after the modification of metal oxide. Therefore the modification should be in such a manner that the wetable property of the metal oxide should not be lost, only reduced.

The modification on the metal oxide can be achieved by chemical reactions in such a way that it forms a stearic hindrance, by coating, covalently binding and polymerization of small molecules to form a net-like structure or by any other chemical reaction or physical change or combination of both that can reduce the density of active Lewis acid centers at the metal oxide. The polymerization coating (2) can be negative or positive charged or of no charge, or combination of two or all three.

Hydrophobic groups containing metal oxide can be in the form of particles, film, coating or any other form, which can be used for the purification of peptides, protein or other molecules. The shape of the said particle is either spherical, broken particles, porous, non porous, chromatographic particles of any shape and size (0.1-10,00000 micron). The said film is mono-layer or multi-layer on a surface and the said surface is porous, solid, and net or any other surface which can be coated with the said film. The thickness of the said film is from mono layer to 10 mm thickness.

The said molecules are bio-molecules such as proteins, peptides, lipids, carbohydrates, nucleic acids, DNA, RNA. The modified metal oxides are Al, Zn, Ti, Zr, Hf, Ga, In and Tl or mixed metal oxides or any other metal oxides of hydrophilic character. Furthermore, the said modified metal oxides are mixed metal oxides of Titanium, Zirconium, Hafnium, Aluminum, Gallium, Indium, transition metal oxides or any metal oxide, which can hydrophilic properties. The mixed metal oxide means a combination of two or more metal oxides.

The metal oxides have said modification by means of chemical reaction, physical method such as coating, pressure temperature; covalent binding or any other chemical or physical process or a combination of both, which reduces the number of Lewis acid centers at the metal oxide. Furthermore, modified metal oxide is coated on the particles such as silica, polymer, porous, nonporous silica or polymer particles.

These modified metal oxides can be used for the chromatography, sample prep, biochip, diagnostics or any other process or production of the biomolecules. Furthermore, these modified metal oxides can be placed in columns for high performance liquid chromatography (HPLC), solid phase extraction column, single column, multiple column, 96, 384, 1536-well plates, film, embedded on the surface.

The said modified metal oxide coated plates can be used for MALDI mass spectrometer.

The hydrophobic modification can be performed during the purification step.

A method for the purification and enrichment of the biomolecules can be developed by using the said modified metal oxide. wherein modified metal oxides have additional modifications, which can be used for the purification of biomolecules, some examples of such additional modifications are as follows (but not limited to) affinity, receptors, enzymes, ligands wherein wet-able means, that the modified metal oxide can be wetted with the aqueous solution, said aqueous solutions are buffers in water, pure water or any other aqueous solution.

Said biomolecules are proteins, peptides, lipids, carbohydrates, DNA, RNA, small bio active molecules, polymers and/or a combination of any two or more, which are covalently bound.

Purification means separation, desalting, purification, concentration.

Modified metal oxide is used for the chromatography, sample prep, biochip, diagnostics or any other process or production of the biomolecules.

The said metal oxide can be placed in columns of high pressure liquid column (HPLC), solid phase extraction column, single column, multiple column, 96, 384, 1536-well plates, film, embedded on the surface.

The said metal oxide coated plates can be used for MALDI mass spectrometer.

### EXAMPLE 1

The random polyethylene (polymer) coated TiO2 (30 um) particles and 30 um C-18 silica particles are used. A peptide mixture obtained after the trypsin digestion of bovine albumin was used. Two mini columns of 50 ul containing TiO2 particle are used, one contains modified TiO2 and other C-18 silica. The binding of peptides is achieved in aqueous solution and after several washes with water the peptides are eluted with 60% acetonitrile. The samples of peptides are analyzed by HPLC and Mass spectrometer (MALDI). The significant difference in the binding of different phosphopeptides was observed.

While a specific embodiment of the invention has been shown and described in detail to illustrate the application of the principles of the invention, it is understood that the invention may be embodied otherwise without departing from such principles and that various modifications, alternate constructions, and equivalents will occur to those skilled in the area given the benefit of this disclosure and the embodiment described herein, as defined by the appended claims.

What is claimed is:

1. A method for the purification and enrichment of biomolecules by using modified metal oxide particles, such particles are hydrophobic and wet able with aqueous solution.

2. A method for the purification and enrichment of biomolecules as in claim 1, wherein the metal oxides are Al, Zn, Ti, Zr, Hf, Ga, In and Tl and In or mixed metal oxides.

3. A method for the purification and enrichment of biomolecules as in claim 1, wherein the said metal oxides modification is performed chemically or physically or a combination of both.

4. A method for the purification and enrichment of biomolecules as in claim 1, wherein modification is performed in such a way that it makes the metal oxide hydrophobic.

5. A method for the purification and enrichment of biomolecules as in claim 1, wherein modified metal oxides have additional modifications, which can be used for the purification of biomolecules, some examples of such additional modifications are as follows (but not limited to) affinity, receptors, enzymes, ligands.

6. A method for the purification and enrichment of biomolecules as in claim 1, wherein metal oxide modification is of random order and not all the oxide surface is covered with the modification.

7. A method for the purification and enrichment of biomolecules as in claim 1, wherein metal oxides based chromatographic media are in the form of particles, which can be spherical or non spherical, porous or non porous or of any shape and size (0.1-100000 micron).

8. A method for the purification and enrichment of biomolecules as in claim 1, wherein a film of metal oxides is coated on a surface, the said surface is porous, solid, and net or any surface which can be coated with the said film.

9. A method for the purification and enrichment of biomolecules as in claim 1, wherein wettable means, the modified metal oxide can be wetted with the aqueous solution, said aqueous solutions are buffers in water, pure water or any other aqueous solution.

10. A method for the purification and enrichment of biomolecules as in claim 1, wherein said biomolecules are proteins, peptides, lipids, carbohydrates, DNA, RNA, small bio active molecules, polymers and/or a combination of any two or more, which are covalently bound.

11. A method for the purification and enrichment of biomolecules as in claim 1, wherein purification means separation, desalting, purification, concentration.

12. A method for the purification and enrichment of biomolecules as in claim 1, wherein said modified metal oxide is used for the chromatography, sample prep, biochip, diagnostics or any other process or production of the biomolecules.

13. A method for the purification and enrichment of biomolecules as in claim 1, wherein said metal oxide can be placed in column, high pressure liquid column (HPLC), solid phase extraction column, single column, multiple column, 96, 384, 1536-well plates, film, embedded on the surface.

14. A method for the purification and enrichment of biomolecules as in claim 1 wherein said metal oxide coated plates can be used for MALDI mass spectrometer.
